(19) United States
(12) Patent Application Publication
(10) Pub. No.: US 2017/0009300 A1
(43) Pub. Date: Jan. 12, 2017

(54) MARKER SEQUENCES FOR RHEUMATOID ARTHRITIS AND USE THEREOF

(71) Applicant: PROTAGEN Aktiengesellschaft, Dortmund (DE)

(72) Inventors: Jens Beator, Kreiensen (DE); Angelika Lüking, Bochum (DE); Axel Kowald, Bochum (DE); Helmut E. Meyer, Recklinghausen (DE)

(21) Appl. No.: 15/274,493

(22) Filed: Sep. 23, 2016

Related U.S. Application Data
(63) Continuation of application No. 14/530,864, filed on Nov. 3, 2014, now abandoned.
Application No. 14/530,864 is a continuation of application No. 12/676,223, filed on Jun. 21, 2010, now abandoned.

(30) Foreign Application Priority Data
Sep. 3, 2007 (DE) 102007041654.9
Sep. 3, 2007 (DE) 102007041656.5

(51) Int. Cl. C12Q 1/68; G01N 33/564

(52) U.S. Cl. CPC C12Q 1/6883; G01N 33/564; C12Q 2600/158; G01N 2800/102; G01N 2800/50

(57) ABSTRACT

The present invention relates to new marker sequences for rheumatoid arthritis and the diagnostic use thereof together with a method for screening of potential active substances for rheumatoid arthritis by means of these marker sequences. Furthermore, the invention relates to a diagnostic device containing such marker sequences for rheumatoid arthritis, in particular a protein biochip and the use thereof.

### RELATED APPLICATIONS

This application is a continuation of patent application Ser. No. 14/530,864 filed on Nov. 3, 2014, which is a continuation of patent application Ser. No. 12/676,223 filed on Mar. 3, 2010, which is a national stage application (under 35 U.S.C. §371) of PCT/DE2008/001547 filed Sep. 3, 2008, which claims benefit of German application 102007041656.5, filed Sep. 3, 2007, and German application, 102007041654.9, filed Sep. 3, 2007. The entire content of each above application is hereby incorporated by reference in its entirety.

### SUBMISSION OF SEQUENCE LISTING

The Sequence Listing associated with this application is filed in electronic format via EFS-Web and hereby incorporated by reference into the specification in its entirety. The name of the text file containing the Sequence Listing is Sequence_Listing_074027_0015_02. The size of the text file is 2,499 KB, and the text file was created on Sep. 23, 2016.

The present invention relates to new marker sequences for rheumatoid arthritis and the diagnostic use thereof together with a method for screening potential active substances for rheumatoid arthritis by means of these marker sequences. Furthermore, the invention relates to a diagnostic device containing marker sequences of this type for rheumatoid arthritis, in particular a protein biochip and the use thereof.

Protein biochips are gaining increasing industrial importance in analysis and diagnosis as well as in pharmaceutical development. Protein biochips have become established as screening instruments.

The rapid and highly parallel detection of a multiplicity of specifically binding analysis molecules in a single experiment is rendered possible hereby. To produce protein biochips, it is necessary to have the required proteins available. For this purpose, in particular protein expression libraries have become established. The high throughput cloning of defined open reading frames is one possibility (Heyman, J. A., Comthwaite, J., Foncerrada, L., Gilmore, J. R., Gontang, E., Hartman, K. J., Hernandez, C. L., Hood, R., Hull, H. M., Lee, W. Y., Marcil, R., Marsh, E. J., Mudd, K. M., Patino, M. J., Purcell, T. J., Rowland, J. J., Sindici, M. L. and Hoeffler, J. P., (1999) Genome-scale cloning and expression of individual open reading frames using topoisomerase I-mediated ligation. Genome Res, 9, 383-392; Kersten, B., Feilner, T., Kramer, A., Wehrmeyer, S., Possling, A., Witt, I., Zanor, M. I., Stracke, R., Lueking, A., Kreutzberger, J., Lehrach, H. and Cahill, D. J. (2003) Generation of Arabidopsis protein chip for antibody and serum screening. Plant Molecular Biology, 52, 999-1010; Reboul, J., Reboul, J., Vaglio, P., Rual, J. F., Lamesch, P., Martinez, M., Armstrong, C M., Li, S., Jacotot, L., Bertin, N., Janky, R., Moore, T., Hudson, J. R., Jr., Hartley, J. L., Brasch, M. A., Vandenhaute, J., Boulton, S., Endress, G. A., Jenna, S., Chevet, E., Papasotiropoulos, V., Tolias, P. P., Ptacek, J., Snyder, M., Huang, R., Chance, M. R., Lee, H., Doucette-Stamm, L., Hill, D. E. and Vidal, M. (2003) C. elegans ORFeome Version 1.1: experimental verification of the genome annotation and resource for proteome-scale protein expression. Nat Genet, 34, 35-41.; Walhout, A. J., Temple, G. F., Brasch, M. A., Hartley, J. L., Lorson, M. A., van den Heuvel, S. and Vidal, M. (2000) GATEWAY recombinational cloning: application to the cloning of large numbers of open reading frames or ORFeomes. Methods Enzymol, 328, 575-592). However, an approach of this type is strongly connected to the progress of the genome sequencing projects and the annotation of these gene sequences. Furthermore, the determination of the expressed sequence can be ambiguous due to differential splicing processes. This problem may be circumvented by the application of cDNA expression libraries (Buessow, K., Cahill, D., Nietfeld, W., Bancroft, D., Scherzinger, E., Lehrach, H. and Walter, G. (1998) A method for global protein expression and antibody screening on high-density filters of an arrayed cDNA library. Nucleic Acids Research, 26, 5007-5008; Buessow, K., Nordhoff, E., Lubbert, C, Lehrach, H. and Walter, G. (2000) A human cDNA library for high-throughput protein expression screening. Genomics, 65, 1-8; Holz, C, Lueking, A., Bovekamp, L., Gutjahr, C, Bolotina, N., Lehrach, H. and Cahill, D. J. (2001) A human cDNA expression library in yeast enriched for open reading frames. Genome Res, 11, 1730-1735; Lueking, A., Holz, C, Gotthold, C, Lehrach, H. and Cahill, D. (2000) A system for dual protein expression in Pichia pastoris and Escherichia coli, Protein Expr. Purif., 20, 372-378).

The cDNA of a particular tissue is hereby cloned into a bacterial or an eukaryotic expression vector, such as, e.g., yeast. The vectors used for the expression are generally characterized in that they carry inducible promoters that may be used to control the time of protein expression. Furthermore, expression vectors have sequences for so-called affinity epitopes or affinity proteins, which on the one hand permit the specific detection of the recombinant fusion proteins by means of an antibody directed against the affinity epitope, and on the other hand the specific purification via affinity chromatography (IMAC) is rendered possible.

For example, the gene products of a cDNA expression library from human fetal brain tissue in the bacterial expression system Escherichia coli were arranged in high-density format on a membrane and could be successfully screened with different antibodies. It was possible to show that the proportion of full-length proteins is at least 66%. Additionally, the recombinant proteins from the library could be expressed and purified in a high-throughput manner (Braun P., Hu, Y., Shen, B., Halleck, A., Koundinya, M., Harlow, E. and LaBaer, J. (2002) Proteome-scale purification of human proteins from bacteria. Proc Natl Acad Sci USA, 99, 2654-2659; Buessow (2000) supra; Lueking, A., Horn, M., Eickhoff, H., Buessow, K., Lehrach, H. and Walter, G. (1999) Protein microarrays for gene expression and antibody screening. Analytical Biochemistry, 270,103-111). Protein biochips of this type based on cDNA expression libraries are in particular the subject matter of WO 99/57311 and WO 99/57312.

Furthermore, in addition to antigen-presenting protein biochips, antibody-presenting arrangements are likewise described (Lal et al (2002) Antibody arrays: An embryonic but rapidly growing technology, DDT, 7, 143-149; Kusnezow et al. (2003), Antibody microarrays: An evaluation of production parameters, Proteomics, 3, 254-264).

However, there is a great need to provide indication-specific diagnostic devices, such as a protein biochip.

Marker sequences and the diagnostic use thereof for rheumatoid arthritis, in particular in the embodiment of a protein biochip, as well as tests in this regard for the screening of active substances have not been described in the prior art.

The object of the present invention is therefore to provide marker sequences and their diagnostic use.

### BRIEF DESCRIPTION OF SEVERAL VIEWS OF THE DRAWING

FIG. 1A and FIG. 1B show the differential screening between two protein biochips from respectively one cDNA expression bank of a patient and a healthy test subject. The differential clones are detected by means of fluorescent labeling and evaluated by means of bioinformatics.

The provision of specific marker sequences permits a reliable diagnosis and stratification of patients with rheumatoid arthritis, in particular by means of a protein biochip.

The invention therefore relates to the use of marker sequences for the diagnosis of rheumatoid arthritis, wherein at least one marker sequence of a cDNA selected from the group SEQ 1-488 or respectively a protein coding therefor or respectively a partial sequence or fragment thereof (hereinafter: marker sequences according to the invention) is determined on or from a patient to be examined.

It was possible to identify the marker sequences according to the invention by means of differential screening of samples from healthy test subjects with patient samples with rheumatoid arthritis.

The term “rheumatoid arthritis (RA)” is defined, e.g., according to Pschyrembel, de Gruyter, 261st edition (2007), Berlin. According to the invention, “juvenile idiopathic arthritis” is likewise covered (ICD-10: MOS.-. Abbr.: JIA. Older synonyms: juvenile rheumatoid arthritis, juvenile chronic arthritis, Morbus Still or more popularly: childhood rheumatism), which a collective term for a number of primarily arthrotopic diseases (arthritis) of the category of rheumatic diseases in childhood (juvenile) (Definition e.g., according to Pschyrembel, de Gruyter, 261st edition (2007), Berlin). This is a polygenic disease, which can be diagnosed particularly advantageously by means of the marker sequences according to the invention, preferably by the marker sequences SEQ 401-488.

In a further embodiment at least 2 to 5 or 10, preferably 30 to 50 marker sequences or 50 to 100 or more marker sequences are determined on or from a patient to be examined.

In a particular embodiment of the invention, the marker sequences of the SEQ 1-20 are particularly preferred, the marker sequences SEQ 21-50 are prefened, and furthermore the marker sequences SEQ 51-100 are preferred.

In a further embodiment of the invention, the marker sequences SEQ 1-10 and SEQ 11-20, as well as preferably SEQ 21-30, SEQ 31-40 or SEQ 41-50 are respectively particularly preferred.

Furthermore preferred are the marker sequences SEQ 401-488 for the diagnosis of juvenile rheumatoid arthritis, in particular SEQ 401-420, SEQ 421-440, SEQ 441-460 and SEQ 461-488.

In a further embodiment of the invention, the marker sequences according to the invention can likewise be combined, supplemented, fused or expanded likewise with known biomarkers for this indication.

In a prefened embodiment, the determination of the marker sequences is carried out outside the human body and the determination is carried out in an ex vivo/in vitro diagnosis.

In a further embodiment of the invention, the invention relates to the use of marker sequences as diagnostic agents, wherein at least one marker sequence of a cDNA is selected from the group SEQ 1-488 or respectively a protein coding therefor or respectively a partial sequence or fragment thereof.

Furthermore, the invention relates to a method for the diagnosis of rheumatoid arthritis, wherein a.) at least one marker sequence of a cDNA selected from the group SEQ 1-488 or respectively a protein coding therefor or respectively a partial sequence or fragment thereof is applied to a solid support and b.) is brought into contact with body fluid or tissue extract of a patient and c.) the detection of an interaction of the body fluid or tissue extract with the marker sequences from a.) is carried out.

The invention therefore likewise relates to diagnostic agents for the diagnosis of rheumatoid arthritis respectively selected from the group SEQ 1-488 or respectively a protein coding therefor or respectively a partial sequence or fragment thereof.

The detection of an interaction of this type can be carried out, for example, by a probe, in particular by an antibody.

The invention therefore likewise relates to the object of providing a diagnostic device or an assay, in particular a protein biochip, which permits a diagnosis or examination for rheumatoid arthritis.

Furthermore, the invention relates to a method for the stratification, in particular risk stratification and/or therapy control of a patient with rheumatoid arthritis, wherein at least one marker sequence of a cDNA selected from the group SEQ 1-488 or respectively a protein coding therefor is determined on a patient to be examined.

Furthermore, the stratification of the patients with rheumatoid arthritis in new or established subgroups of rheumatoid arthritis is also covered, as well as the expedient selection of patient groups for the clinical development of new therapeutic agents. The term therapy control likewise covers the allocation of patients to responders and nonresponders regarding a therapy or the therapy course thereof.

“Diagnosis” for the purposes of this invention means the positive determination of rheumatoid arthritis by means of the marker sequences according to the invention as well as the assignment of the patients to rheumatoid arthritis. The term diagnosis covers medical diagnostics and examinations in this regard, in particular in-vitro diagnostics and laboratory diagnostics, likewise proteomics and nucleic acid blotting. Further tests can be necessary to be sure and to exclude other diseases. The term diagnosis therefore likewise covers the differential diagnosis of rheumatoid arthritis by means of the marker sequences according to the invention and the prognosis of rheumatoid arthritis.

“Stratification or therapy control” for the purposes of this invention means that the method according to the invention renders possible decisions for the treatment and therapy of the patient, whether it is the hospitalization of the patient, the use, effect and/or dosage of one or more drugs, a therapeutic measure or the monitoring of a course of the disease and the course of therapy or etiology or classification of a disease, e.g., into a new or existing subtype or the differentiation of diseases and the patients thereof.

In a further embodiment of the invention, the term “stratification” covers in particular the risk stratification with the prognosis of an outcome of a negative health event.

Within the scope of this invention, “patient” means any test subject—human or mammal—with the proviso that the test subject is tested for rheumatoid arthritis.

The term “marker sequences” for the purposes of this invention means that the cDNA or the polypeptide or protein that can be respectively obtained therefrom are significant for rheumatoid arthritis. For example, the cDNA or the polypeptide or protein that can be respectively obtained therefrom can exhibit an interaction with substances from the body fluid or tissue extract of a patient with rheumatoid arthritis (e.g., antigen (epitope)/antibody (paratope) interaction). For the purposes of the invention “wherein at least one marker sequence of a cDNA selected from the group SEQ 1-488 or respectively a protein coding therefor or respectively a partial sequence or fragment thereof is determined on a patient to be examined” means that an interaction between the body fluid or tissue extract of a patient and the marker sequences according to the invention is detected. An interaction of this type is, e.g., a bond, in particular a binding substance on at least one marker sequence according to the invention or in the case of a cDNA the hybridization with a suitable substance under selected conditions, in particular stringent conditions (e.g., such as usually defined in J. Sambrook, E. F. Fritsch, T. Maniatis (1989), Molecular cloning: A laboratory manual, 2nd Edition, Cold Spring Harbor Laboratory Press, Cold Spring Harbor, USA or Ausubel, “Current Protocols in Molecular Biology,” Green Publishing Associates and Wiley Interscience, N.Y. (1989)). One example of stringent hybridization conditions is: hybridization in 4×SSC at 65° C. (alternatively in 50% formamide and 4×SSC at 42° C.), followed by several washing steps in 0.1×SSC at 65° C. for a total of approximately one hour. An example of less stringent hybridization conditions is hybridization in 4×SSC at 37° C., followed by several washing steps in 1×SSC at room temperature.

According to the invention, substances of this type are constituents of a body fluid, in particular blood, whole blood, blood plasma, blood serum, patient serum, urine, cerebrospinal fluid, synovial fluid or of a tissue extract of the patient.

In a further embodiment of the invention, however, the marker sequences according to the invention can be present in a significantly higher or lower expression rate or concentration that indicates rheumatoid arthritis. The relative sick/healthy expression rates of the marker sequences for rheumatoid arthritis according to the invention are hereby determined by means of proteomics or nucleic acid blotting.

In a further embodiment of the invention, the marker sequences have a recognition signal that is addressed to the substance to be bound (e.g., antibody, nucleic acid). It is preferred according to the invention for a protein the recognition signal is an epitope and/or a paratope and/or a hapten and for a cDNA is a hybridization or binding region.

The marker sequences according to the invention are the subject matter of Table A and can be clearly identified by the respectively cited database entry (also by means of the Internet: http://www.ncbi.nlm.nih.gov/) (see in Table A: accession no. there).

According to the invention, the marker sequences also cover those modifications of the cDNA sequence and the corresponding amino acid sequence as chemical modification, such as citrullination, acetylation, phosphorylation, glycosylation or poly(A) strand and other modifications known to one skilled in the art.

In a further embodiment of the invention, partial sequences or fragments of the marker sequences according to the invention are likewise covered. In particular those partial sequences that have an identity of 95%, 90%, in particular 80% or 70% with the marker sequences according to the invention.

In a further embodiment, the respective marker sequence can be represented in different quantities in one more regions on a solid support. This permits a variation of the sensitivity. The regions can have respectively a totality of marker sequences, i.e., a sufficient number of different marker sequences, in particular 2 to 5 or 10 or more and optionally more nucleic acids and/or proteins, in particular biomarkers. However, at least 96 to 25,000 (numerical) or more from different or identical marker sequences and further nucleic acids and/or proteins, in particular biomarkers are preferred. Furthermore preferred are more than 2,5000, in particular preferred 10,000 or more different or identical marker sequences and optionally further nucleic acids and/or proteins, in particular biomarkers.

Another object of the invention relates to an arrangement of marker sequences containing at least one marker sequence of a cDNA selected from the group SEQ 1-488 or respectively a protein coding therefor. Preferably, the arrangement contains at least 2 to 5 or 10, preferably 30 to 50 marker sequences or 50 to 100 or more marker sequences.

Within the scope of this invention, “arrangement” is synonymous with “array,” and if this “array” is used to identify substances on marker sequences, this is to be understood to be an “assay” or diagnostic device. In a preferred embodiment, the arrangement is designed such that the marker sequences represented on the arrangement are present in the form of a grid on a solid support. Furthermore, those arrangements are preferred that permit a high-density arrangement of protein binders and the marker sequences are spotted. Such high-density spotted arrangements are disclosed, for example, in WO 99/57311 and WO 99/57312 and can be used advantageously in a robot-supported automated high throughput method.

Within the scope of this invention, however, the term “assay” or diagnostic device likewise comprises those embodiments of a device, such as ELISA (e.g., individual wells of a microtiter plate are coated with the marker sequences or combinations of marker sequences according to the invention, optionally applied in a robot-supported manner in the individual wells of the microtiter plate; examples are diagnostic ELISA kits by Phadia or “Searchlight” multiplex ELISA kits by Pierce/Thermo Fisher Scientific), bead-based assay (spectrally distinguishable bead populations are coated with marker sequences/combinations of marker sequences. The patient sample is incubated with this bead population and bound (auto) antibodies are detected by means of a further fluorescence-labeled secondary antibody/detection reagent via measurement of the fluorescence; i.e., Borrelia IgG kit or Athena Multilyte by Multimetrix), line assay (marker sequences according to the invention or combinations of marker sequences are immobilized on membranes in a robot-supported manner, which are examined/incubated with the patient sample; example “Euroline” by Euroimmun AG), Western Blot (example “Euroline-WB” by Euroimmun AG), immunochromatographic methods (e.g., lateral flow immunoassays; marker sequences/combinations of marker sequences are immobilized on test strips (membranes, U.S. Pat. No. 5,714,389 and the like); example “One Step HBsAg” test device by Aeon Laboratories) or similar immunological single or multiplex detection measures.

The marker sequences of the arrangement are fixed on a solid support, but preferably spotted or immobilized even printed on, i.e. applied in a reproducible manner. One or more marker sequences can be present multiple times in the totality of all marker sequences and present in different quantities based on one spot. Furthermore, the marker sequences can be standardized on the solid support (i.e., by means of serial dilution series of, e.g., human globulins as internal calibrators for data normalization and quantitative evaluation).

The invention therefore relates to an assay or a protein biochip comprising an arrangement containing marker sequences according to the invention.

In a further embodiment, the marker sequences are present as clones. Clones of this type can be obtained, for example, by means of a cDNA expression library according to the invention (Buessow et al. 1998 (supra)). In a preferred embodiment, such expression libraries containing clones are obtained using expression vectors from a cDNA expression library comprising the cDNA marker sequences. These expression vectors preferably contain inducible promoters. The induction of the expression can be carried out, e.g., by means of an inductor, such as IPTG. Suitable expression vectors are described in Terpe et al . (Terpe T Appl Microbiol Biotechnol. 2003 January; 60(5) : 523-33).

One skilled in the art is familiar with expression libraries, they can be produced according to standard works, such as Sambrook et al, “Molecular Cloning, A laboratory handbook, 2nd edition” (1989), CSH press, Cold Spring Harbor, New York. Expression libraries are also preferred which are tissue-specific (e.g., human tissue, in particular human organs). Furthermore included according to the invention are expression libraries that can be obtained by exon-trapping. A synonym for expression library is expression bank.

Also preferred are protein biochips or corresponding expression libraries that do not exhibit any redundancy (so-called: Uniclone® library) and that may be produced, for example, according to the teachings of WO 99/57311 and WO 99/57312. These preferred Uniclone libraries have a high portion of non-defective fully expressed proteins of a cDNA expression library.

Within the context of this invention, the clones can also be, but not limited to, transformed bacteria, recombinant phages or transformed cells from mammals, insects, fungi, yeasts or plants.

The clones are fixed, spotted or immobilized on a solid support.

The invention therefore relates to an arrangement wherein the marker sequences are present as clones.

Additionally, the marker sequences can be present in the respective form of a fusion protein, which contains, for example, at least one affinity epitope or tag. The tag may be one such as contains c-myc, his tag, arg tag, FLAG, alkaline phosphatase, VS tag, T7 tag or strep tag, HAT tag, NusA, S tag, SBP tag, thioredoxin, DsbA, a fusion protein, preferably a cellulose-binding domain, green fluorescent protein, maltose-binding protein, calmodulin-binding protein, glutathione S-transferase or lacZ.

A marker sequence can also be composed of several individual marker sequences. This can comprise the cloning of individual fragments to form a large common fragment and the expression of this combined fragment.

In all of the embodiments, the term “solid support” covers embodiments such as a filter, a membrane, a magnetic or fluorophore-labeled bead, a silica wafer, glass, metal, ceramics, plastics, a chip, a target for mass spectrometry or a matrix. However, a filter is preferred according to the invention.

As a filter, furthermore PVDF, nitrocellulose or nylon is preferred (e.g., Immobilon P Millipore, Protran Whatman, Hybond N+ Amersham).

In another preferred embodiment of the arrangement according to the invention, the arrangement corresponds to a grid with the dimensions of a microtiter plate (8-12 wells strips, 96 wells, 384 wells or more), a silica wafer, a chip, a target for mass spectrometry, or a matrix.

In a further embodiment, the invention relates to an assay or a protein biochip for identifying and characterizing a substance for rheumatoid arthritis, characterized in that an arrangement or assay according to the invention is a.) brought into contact with at least one substance to be tested and b.) a binding success is detected.

Furthermore, the invention relates to a method for identifying and characterizing a substance for rheumatoid arthritis, characterized in that an arrangement or assay according to the invention is a.) brought into contact with at least one substance to be tested and b.) a binding success is detected.

The substance to be tested can be any native or non-native biomolecule, a synthetic chemical molecule, a mixture or a substance library.

After the substance to be tested contacts a marker sequence, the binding success is evaluated, which, for example, is carried out using commercially available image analyzing software (GenePix Pro (Axon Laboratories), Aida (Ray test), ScanArray (Packard Bioscience).

The visualization of protein-protein interactions according to the invention (e.g., protein on marker sequence, as antigen/antibody) or corresponding “means for detecting the binding success” can be performed, for example, using fluorescence labeling, biotinylation, radioisotope labeling or colloid gold or latex particle labeling in the usual way. A detection of bound antibodies is carried out with the aid of secondary antibodies, which are labeled with commercially available reporter molecules (e.g., Cy, Alexa, Dyomics, FITC or similar fluorescent dyes, colloidal gold or latex particles), or with reporter enzymes, such as alkaline phosphatase, horseradish peroxidase, etc., and the corresponding colorimetric, fluorescent or chemiluminescent substrates. Readout is conducted, e.g., using a microarray laser scanner, a CCD camera or visually.

In a further embodiment, the invention relates to a drug/active substance or prodrug developed for rheumatoid arthritis and obtainable through the use of the assay or protein biochip according to the invention.

The invention therefore likewise relates to the use of an arrangement according to the invention or an assay for screening active substances for rheumatoid arthritis.

In a further embodiment, the invention therefore likewise relates to a target for the treatment and therapy of rheumatoid arthritis respectively selected from the group SEQ 1-488 or a protein respectively coding therefor.

In a further embodiment, the invention likewise relates to the use of the marker sequences according to the invention, preferably in the form of an arrangement, as an affinity material for carrying out an apheresis or in the broadest sense a blood lavage, wherein substances from body fluids of a patient with rheumatoid arthritis, such as blood or plasma, bind to the marker sequences according to the invention and consequently can be selectively withdrawn from the body fluid.

Examples and Figures:

Ten or more patient samples were individually screened against a cDNA expression library. The rheumatoid arthritis -specific expression clones were determined through a comparison with ten or more healthy samples. The identity of the marker sequences was determined by DNA sequencing.

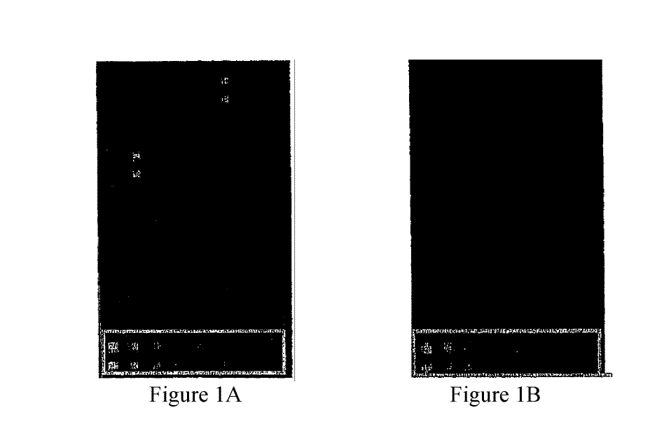

FIG. 1A and FIG. 1B show the differential screening between two protein biochips from respectively one cDNA expression bank of a patient and a healthy test subject. The differential clones are detected by means of fluorescent labeling and evaluated by means of bioinformatics.

In Table A, column “Nr” refers to the number of the sequence identifier of a cDNA marker sequence in the Sequence Listing. For example, Nr 50 refers to marker sequence SEQ ID NO: 50 in the Sequence Listing.

TABLE A

| Nr | PRI | Accsssion No |
|---|---|---|
| 1 | A | gi\|33519473 |
| 2 | A | gi\|33469975 |
| 3 | A | gi\|113421166 |
| 4 | A | gi\|113411825 |
| 5 | A | gi\|55925645 |
| 6 | A | gi\|31341967 |
| 7 | A | gi\|37537717 |
| 8 | A | gi\|51464299 |
| 9 | A | gi\|31343485 |
| 10 | A | gi\|73622128 |
| 11 | A | gi\|22202618 |
| 12 | A | gi\|21956639 |
| 13 | A | gi\|47894110 |
| 14 | A | gi\|74048536 |
| 15 | A | gi\|39573729 |
| 16 | A | gi\|113421846 |
| 17 | A | gi\|34098945 |
| 18 | A | gi\|30583601 |
| 19 | A | NM_012292 |
| 20 | A | NM_004499 |
| 21 | B | 61064_8_E11 |
| 22 | B | gi\|83716023 |
| 23 | B | NM_006796 |
| 24 | B | gi\|11386138 |
| 25 | B | gi\|21389576 |
| 26 | B | gi\|56676308 |
| 27 | B | gi\|4503744 |
| 28 | B | gi\|57222567 |
| 29 | B | gi\|7512821 |
| 30 | B | NM_000973 |
| 31 | B | gi\|17149837 |
| 32 | B | NM_002954 |
| 33 | B | gi\|22219473 |
| 34 | B | gi\|30583735 |
| 35 | B | gi\|53733398 |
| 36 | B | gi\|89057118 |
| 37 | B | gi\|12804481 |
| 38 | B | NM_003768 |
| 39 | B | gi\|46391095 |
| 40 | B | NW_926918 |
| 41 | B | gi\|89041118 |
| 42 | B | gi\|14424731 |
| 43 | B | gi\|30410780 |
| 44 | B | NM_005707 |
| 45 | B | gi\|4758937 |
| 46 | B | gi\|7661695 |
| 47 | B | gi\|13559175 |
| 48 | B | gi\|83367078 |
| 49 | B | gi\|48146439 |
| 50 | B | gi\|33591068 |
| 51 | C | gi\|34850060 |
| 52 | C | gi\|34147654 |
| 53 | C | gi\|62526046 |
| 54 | C | gi\|52545622 |
| 55 | C | gi\|7512499 |
| 56 | C | gi\|40255020 |
| 57 | C | gi\|46389548 |
| 58 | C | gi\|2911264 |
| 59 | C | gi\|15431289 |
| 60 | C | 61064_8_H12 |
| 61 | C | gi\|13569612 |
| 62 | C | gi\|38679891 |
| 63 | C | gi\|88943682 |
| 64 | C | gi\|5381417 |
| 65 | C | gi\|4504982 |
| 66 | C | gi\|5453690 |
| 67 | C | NM_020713 |
| 68 | C | gi\|21748598 |
| 69 | C | NM_005354 |
| 70 | C | NM_002473 |
| 71 | C | gi\|3287489 |
| 72 | C | gi\|61656605 |
| 73 | C | gi\|68800242 |
| 74 | C | gi\|21620021 |
| 75 | C | gi\|20149616 |
| 76 | C | gi\|40226207 |
| 77 | C | gi\|40807483 |
| 78 | C | NM_003475 |
| 79 | C | gi\|61966904 |
| 80 | C | NM_001009998 |
| 81 | C | gi\|42490757 |
| 82 | C | gi\|34335231 |
| 83 | C | gi\|11545906 |
| 84 | C | gi\|7245833 |
| 85 | C | gi\|7657677 |
| 86 | C | NM_000477 |
| 87 | C | gi\|39654744 |
| 88 | C | gi\|13938597 |
| 89 | C | gi\|33874730 |
| 90 | C | gi\|19743569 |
| 91 | C | gi\|37544107 |
| 92 | C | gi\|51468814 |
| 93 | C | gi\|21739976 |
| 94 | C | gi\|4758219 |
| 95 | C | NM_004559 |
| 96 | C | gi\|5689527 |
| 97 | C | gi\|31077184 |
| 98 | C | gi\|24308369 |
| 99 | C | gi\|56203109 |
| 100 | C | gi\|4507398 |
| 101 | D | gi\|17981697 |
| 102 | D | gi\|32129198 |
| 103 | D | gi\|6912539 |
| 104 | D | gi\|89030746 |
| 105 | D | NM_000386 |
| 106 | D | gi\|20336766 |
| 107 | D | gi\|16306505 |
| 108 | D | gi\|7619703 |
| 109 | D | gi\|253706 |
| 110 | D | gi\|19913395 |
| 111 | D | gi\|33636763 |
| 112 | D | gi\|66346709 |
| 113 | D | gi\|38197056 |
| 114 | D | gi\|29893564 |
| 115 | D | gi\|1362855 |
| 116 | D | gi\|89057343 |
| 117 | D | gi\|50592995 |
| 118 | D | gi\|71361681 |
| 119 | D | gi\|32455265 |
| 120 | D | gi\|10439788 |
| 121 | D | gi\|31092 |
| 122 | D | gi\|113428396 |
| 123 | D | gi\|7705480 |
| 124 | D | gi\|5830438 |
| 125 | D | NT_010194 |
| 126 | D | gi\|179955 |
| 127 | D | gi\|2547076 |
| 128 | D | gi\|4502846 |
| 129 | D | gi\|83641894 |
| 130 | D | gi\|3642665 |
| 131 | D | gi\|3293553 |
| 132 | D | NM_003130 |
| 133 | D | gi\|113431093 |
| 134 | D | gi\|34147660 |
| 135 | D | gi\|85681028 |
| 136 | D | gi\|17572803 |
| 137 | D | gi\|13124797 |
| 138 | D | gi\|83656780 |
| 139 | D | gi\|39725676 |
| 140 | D | gi\|19526471 |
| 141 | D | gi\|13376797 |
| 142 | D | gi\|15214478 |
| 143 | D | 61064_8_H06 |
| 144 | D | gi\|66346647 |
| 145 | D | gi\|32879857 |
| 146 | D | gi\|40889757 |
| 147 | D | gi\|71772259 |
| 148 | D | gi\|51473210 |
| 149 | D | gi\|15680208 |
| 150 | D | gi\|16306717 |
| 151 | D | gi\|4759097 |
| 152 | D | gi\|56550050 |
| 153 | D | gi\|4506903 |
| 154 | D | gi\|10567816 |
| 155 | D | gi\|4758985 |
| 156 | D | gi\|16740583 |
| 157 | D | gi\|1487948 |
| 158 | D | gi\|23238257 |
| 159 | D | gi\|21758184 |
| 160 | D | gi\|56205191 |
| 161 | D | gi\|83641890 |
| 162 | D | gi\|17380594 |
| 163 | D | NM_001025598 |
| 164 | D | NM_001024807 |
| 165 | D | gi\|49456343 |
| 166 | D | gi\|33150630 |
| 167 | D | gi\|21595329 |
| 168 | D | gi\|13124696 |
| 169 | D | gi\|6716561 |
| 170 | D | gi\|25777682 |
| 171 | D | gi\|18426896 |
| 172 | D | gi\|42544170 |
| 173 | D | gi\|30584255 |
| 174 | D | gi\|26249286 |
| 175 | D | 61064_8_C07 |
| 176 | D | gi\|12232414 |
| 177 | D | gi\|4504618 |
| 178 | D | gi\|39645205 |
| 179 | D | NM_004960 |
| 180 | D | gi\|22212941 |
| 181 | D | gi\|345836 |
| 182 | D | gi\|88999578 |
| 183 | D | gi\|27807403 |
| 184 | D | gi\|17386088 |
| 185 | D | gi\|7524353 |
| 186 | D | gi\|5031931 |
| 187 | D | gi\|40789265 |
| 188 | D | gi\|32490572 |
| 189 | D | gi\|14250530 |
| 190 | D | gi\|46249758 |
| 191 | D | gi\|4507557 |
| 192 | D | gi\|547749 |
| 193 | D | gi\|62897169 |
| 194 | D | gi\|9651486 |
| 195 | D | gi\|37182091 |
| 196 | D | gi\|89059027 |
| 197 | D | gi\|34785019 |
| 198 | D | NM_005572 |
| 199 | D | gi\|113428589 |
| 200 | D | gi\|51471030 |
| 201 | D | gi\|51470970 |
| 202 | D | gi\|20987263 |
| 203 | D | gi\|13623595 |
| 204 | D | NM_020967 |
| 205 | D | NM_020529 |
| 206 | D | gi\|34784912 |
| 207 | D | gi\|38014003 |
| 208 | D | gi\|40807365 |
| 209 | D | gi\|182118 |
| 210 | D | gi\|60552339 |
| 211 | D | gi\|33598947 |
| 212 | D | gi\|32401423 |
| 213 | D | gi\|10434157 |
| 214 | D | gi\|1082338 |
| 215 | D | gi\|340219 |
| 216 | D | gi\|31542761 |
| 217 | D | gi\|17149845 |
| 218 | D | gi\|30583065 |
| 219 | D | gi\|38505154 |
| 220 | D | gi\|19923366 |
| 221 | D | gi\|15928941 |
| 222 | D | gi\|18426915 |
| 223 | D | gi\|505108 |
| 224 | D | gi\|34452717 |
| 225 | D | gi\|6855633 |
| 226 | D | gi\|53729342 |
| 227 | D | gi\|224530 |
| 228 | D | gi\|6912602 |
| 229 | D | gi\|40789071 |
| 230 | D | gi\|51706338 |
| 231 | D | gi\|7262378 |
| 232 | D | gi\|34147665 |
| 233 | D | NM_002228 |
| 234 | D | gi\|22713422 |
| 235 | D | gi\|4505904 |
| 236 | D | gi\|16579885 |
| 237 | D | gi\|47078237 |
| 238 | D | gi\|3387977 |
| 239 | D | gi\|88972371 |
| 240 | D | gi\|2981764 |
| 241 | D | gi\|55959290 |
| 242 | D | gi\|89059359 |
| 243 | D | gi\|32425497 |
| 244 | D | gi\|31317308 |
| 245 | D | gi\|77404355 |
| 246 | D | gi\|32880093 |
| 247 | D | gi\|12232384 |
| 248 | D | gi\|38683849 |
| 249 | D | gi\|9966764 |
| 250 | D | gi\|18390331 |
| 251 | D | gi\|30582607 |
| 252 | D | gi\|31543190 |
| 253 | D | gi\|55959087 |
| 254 | D | gi\|7110641 |
| 255 | D | gi\|2632247 |
| 256 | D | gi\|71594 |
| 257 | D | gi\|46370065 |
| 258 | D | gi\|339685 |
| 259 | D | gi\|33869643 |
| 260 | D | gi\|51036581 |
| 261 | D | gi\|10439217 |
| 262 | D | gi\|39725631 |
| 263 | D | gi\|31563519 |
| 264 | D | gi\|31542269 |
| 265 | D | gi\|22477334 |
| 266 | D | gi\|13699813 |
| 267 | D | gi\|51493052 |
| 268 | D | gi\|4503580 |
| 269 | D | gi\|4557839 |
| 270 | D | gi\|39573730 |
| 271 | D | gi\|89059606 |
| 272 | D | gi\|31652250 |
| 273 | D | gi\|47519746 |
| 274 | D | gi\|33244031 |
| 275 | D | gi\|10434039 |
| 276 | D | gi\|57242773 |
| 277 | D | gi\|21704282 |
| 278 | D | gi\|11342680 |
| 279 | D | gi\|30584609 |
| 280 | D | gi\|21739862 |
| 281 | D | gi\|55959475 |
| 282 | D | gi\|42476191 |
| 283 | D | gi\|34533094 |
| 284 | D | gi\|15431301 |
| 285 | D | gi\|26986533 |
| 286 | D | gi\|8922332 |
| 287 | D | gi\|40787650 |
| 288 | D | gi\|9873442 |
| 289 | D | gi\|50086623 |
| 290 | D | gi\|34147350 |
| 291 | D | gi\|12056467 |
| 292 | D | gi\|55925607 |
| 293 | D | gi\|38570091 |
| 294 | D | gi\|29476902 |
| 295 | D | gi\|40796182 |
| 296 | D | gi\|7770137 |
| 297 | D | gi\|113430465 |
| 298 | D | gi\|89040669 |
| 299 | D | gi\|10518498 |
| 300 | D | gi\|34855930 |
| 301 | D | gi\|186696 |
| 302 | D | gi\|21614499 |
| 303 | D | gi\|3192917 |
| 304 | D | gi\|32306539 |
| 305 | D | gi\|54607123 |
| 306 | D | gi\|52856410 |
| 307 | D | gi\|33286445 |
| 308 | D | gi\|26344686 |
| 309 | D | gi\|42716279 |
| 310 | D | gi\|381964 |
| 311 | D | gi\|46852169 |
| 312 | D | gi\|31874210 |
| 313 | D | gi\|71565157 |
| 314 | D | gi\|7705475 |
| 315 | D | gi\|12803375 |
| 316 | D | gi\|113417847 |
| 317 | D | gi\|14110410 |
| 318 | D | gi\|55957624 |
| 319 | D | gi\|89027401 |
| 320 | D | gi\|13435438 |
| 321 | D | gi\|18490263 |
| 322 | D | gi\|4757715 |
| 323 | D | gi\|12804441 |
| 324 | D | gi\|2134743 |
| 325 | D | gi\|6005923 |
| 326 | D | gi\|6841318 |
| 327 | D | gi\|12711674 |
| 328 | D | gi\|31563378 |
| 329 | D | gi\|51173146 |
| 330 | D | gi\|93141017 |
| 331 | D | gi\|23396512 |
| 332 | D | gi\|55961048 |
| 333 | D | gi\|18314624 |
| 334 | D | gi\|27552770 |
| 335 | D | gi\|50345985 |
| 336 | D | gi\|1710248 |
| 337 | D | gi\|7657441 |
| 338 | D | gi\|40226068 |
| 339 | D | gi\|42490910 |
| 340 | D | gi\|21307630 |
| 341 | D | gi\|133254 |
| 342 | D | gi\|340019 |
| 343 | D | gi\|57997038 |
| 344 | D | gi\|40254816 |
| 345 | D | gi\|27436949 |
| 346 | D | gi\|56789232 |
| 347 | D | gi\|38257139 |
| 348 | D | 61064_8_A09 |
| 349 | D | gi\|13929434 |
| 350 | D | NM_001012 |
| 351 | D | gi\|31657179 |
| 352 | D | gi\|16273176 |
| 353 | D | gi\|14165264 |
| 354 | D | gi\|5123454 |
| 355 | D | gi\|24234719 |
| 356 | D | gi\|10720282 |
| 357 | D | gi\|88966845 |
| 358 | D | NM_014497 |
| 359 | D | gi\|40795668 |
| 360 | D | gi\|22538467 |
| 361 | D | gi\|4503179 |
| 362 | D | gi\|68299771 |
| 363 | D | gi\|62896661 |
| 364 | D | gi\|22027479 |
| 365 | D | gi\|41055203 |
| 366 | D | gi\|4758515 |
| 367 | D | gi\|21757045 |
| 368 | D | NM_006086 |
| 369 | D | gi\|4507284 |
| 370 | D | gi\|4502004 |
| 371 | D | gi\|51465675 |
| 372 | D | gi\|14249144 |
| 373 | D | gi\|2276396 |
| 374 | D | gi\|21361525 |
| 375 | D | gi\|34328690 |
| 376 | D | gi\|13177775 |
| 377 | D | gi\|13325058 |
| 378 | D | gi\|1903190 |
| 379 | D | gi\|23111046 |
| 380 | D | NM_006360 |
| 381 | D | gi\|7512569 |
| 382 | D | gi\|50843811 |
| 383 | D | gi\|113423859 |
| 384 | D | gi\|78190466 |
| 385 | D | gi\|7657649 |
| 386 | D | gi\|30583811 |
| 387 | D | gi\|14150165 |
| 388 | D | gi\|31805540 |
| 389 | D | gi\|34289 |
| 390 | D | gi\|46249395 |
| 391 | D | gi\|22137524 |
| 392 | D | gi\|6226705 |
| 393 | D | NM_004494 |
| 394 | D | gi\|37552371 |
| 395 | D | gi\|10241759 |
| 396 | D | NM_015190 |
| 397 | D | gi\|40353728 |
| 398 | D | gi\|135412 |
| 399 | D | 61064_8_F10 |
| 400 | D | gi\|68800343 |
| 401 | E | NW_923984 |
| 402 | E | NM_018442 |
| 403 | E | NM_032281 |
| 404 | E | NM_005778 |
| 405 | E | NM_014859 |
| 406 | E | NM_006352 |
| 407 | E | NM_022088 |
| 408 | E | NM_000516 |
| 409 | E | NM_000237 |
| 410 | E | NM_020825 |
| 411 | E | NM_000076 |
| 412 | E | NM_015720 |
| 413 | E | NM_017596 |
| 414 | E | NM_003195 |
| 415 | E | NM_001280 |
| 416 | E | NM_001704 |
| 417 | E | NM_001686 |
| 418 | E | NM_152704 |
| 419 | E | NT_004350 |
| 420 | E | NM_014680 |
| 421 | E | NM_005801 |
| 422 | E | NM_080390 |
| 423 | E | NT_033903 |
| 424 | E | NM_003025 |
| 425 | E | NM_006036 |
| 426 | E | NM_001551 |
| 427 | E | NM_004380 |
| 428 | E | NM_138559 |
| 429 | E | NM_006352 |
| 430 | E | NM_006428 |
| 431 | E | NT_029419 |
| 432 | E | NW_927628 |
| 433 | E | NM_006353 |
| 434 | E | NM_002154 |
| 435 | E | NM_003025 |
| 436 | E | NM_022359 |
| 437 | E | NM_032514 |
| 438 | E | NW_927195 |
| 439 | E | NM_012295 |
| 440 | E | NW_927628 |
| 441 | E | NM_006958 |
| 442 | E | NM_002013 |
| 443 | E | NM_198943 |
| 444 | E | NM_002256 |
| 445 | E | NM_001098 |
| 446 | E | NM_005225 |
| 447 | E | NM_004712 |
| 448 | E | NT_010641 |
| 449 | E | NM_022730 |
| 450 | E | NM_000934 |
| 451 | E | NM_006590 |
| 452 | E | NT_037887 |
| 453 | E | NM_005736 |
| 454 | E | NM_181697 |
| 455 | E | NM_030907 |
| 456 | E | NM_002613 |
| 457 | E | NM_002013 |
| 458 | E | NM_006373 |
| 459 | E | NM_000969 |
| 460 | E | NM_178159 |
| 461 | E | NM_024671 |
| 462 | E | NW_927762 |
| 463 | E | NM_007029 |
| 464 | E | XM_937970 |
| 465 | E | NM_001031735 |
| 466 | E | NM_001069 |
| 467 | E | NM_006841 |
| 468 | E | NM_000477 |
| 469 | E | NM_203346 |
| 470 | E | NM_012398 |
| 471 | E | NM_005851 |
| 472 | E | NM_023071 |
| 473 | E | NT_005612 |
| 474 | E | NM_006640 |
| 475 | E | NM_016300 |
| 476 | E | NM_182565 |
| 477 | E | NT_079595 |
| 478 | E | NM_025203 |
| 479 | E | NM_014593 |
| 480 | E | NM_033647 |
| 481 | E | NM_001098 |
| 482 | E | NM_000801 |
| 483 | E | NM_001032396 |
| 484 | E | NT_006081 |
| 485 | E | NM_018287 |
| 486 | E | NM_023940 |
| 487 | E | NM_002751 |
| 488 | E | NT_037887 |

We claim:

1. A method for the diagnosis or risk stratification of rheumatoid arthritis comprising detecting an interaction between a body fluid or tissue extract of a patient and least one marker sequence of a cDNA selected from the group consisting of SEQ ID NO: 1-488 or a protein coding therefor, wherein detection of an interaction indicates the presence of rheumatoid arthritis in said patient.

2. An arrangement of marker sequences comprising the marker sequences of the group SEQ ID NO: 1-488.

3. The arrangement according to claim 3, wherein the marker sequences are present as clones.

4. The arrangement according to claim 3, wherein the marker sequences are present on a solid support.

5. A method of apheresis or blood lavage, comprising contacting body fluid of a patient with the arrangement of marker sequences comprising the marker sequences of the group SEQ ID NO: 1-488.
